(19) United States
(12) Patent Application Publication
(10) Pub. No.: US 2023/0389522 A1
(43) Pub. Date: Dec. 7, 2023

(54) NECKLACE BELT FOR PET

(71) Applicants: MAX.K KOREA Inc., Seobusaet-gil (KR); Max K LLC, Houston, TX (US)

(72) Inventors: Kibun Max KOO, Houston, TX (US); Sang Min AN, Seoul (KR)

(73) Assignees: MAX.K KOREA Inc., Seobusaet-gil (KR); Max K LLC, Houston, TX (US)

(21) Appl. No.: 18/183,696

(22) Filed: Mar. 14, 2023

(30) Foreign Application Priority Data
Jun. 3, 2022 (KR) 10-2022-0068362

(51) Int. Cl. A01K 27/00; A01K 29/00

(52) U.S. Cl. CPC A01K 27/001; A01K 29/005

(57) ABSTRACT

The present disclosure relates to a necklace belt for a pet. More particularly, when a wearable sensor device for monitoring a bio-signal of the pet is coupled to the necklace belt for a pet and the necklace belt is worn around the pet, the necklace belt is easily and solidly bent according to a form of a body part of the pet, locked, not overturned on the body part of the pet, and not released even with repeated movements.

### CROSS REFERENCE TO RELATED APPLICATION

This application claims priority to Korea Patent Application No. 10-2022-0068362 filed on Jun. 3, 2022, the content of which is expressly incorporated by reference in its entirety.

### TECHNICAL FIELD

The present disclosure relates to a necklace belt for a pet. More particularly, when a wearable sensor device for monitoring a bio-signal of a pet is coupled to the necklace belt for a pet and the necklace belt is worn around a body part of the pet, the necklace belt is easily and solidly bent according to a form of a body part of the pet, locked, not overturned on the body part of the pet, and not released even with repeated movements.

### BACKGROUND

Recently, growth of nuclear families and single households induces sharp increase in people raising various pets (i.e., companion animals) such as cats or dogs for emotional enhancing or mental health of people. Accordingly, it has become common for people and pets to go for walks together, and interest in a physical condition of pets is greatly increasing.

As the most common method for checking a physical condition of a pet, a sensor device that measures biometric information (e.g., body temperature) of the pet is attached to a body part of the pet, and the owner of the pet receives and checks the biometric information measured by the sensor device via a smartphone of the owner. Accordingly, the owner of the pet can immediately check whether or not abnormality occurs with the pet, and can take immediate countermeasures as soon as the abnormality is discovered. However, a belt is used to attach the sensor device to the body part of the pet. Due to frequent movement of the pet, the belt may be frequently released or be separated from the sensor device, and there are many inconveniences in use, such as being overturned on the body part of the pet. In addition, when the belt is made of hard plastic or leather, trouble may occur at a part of the skin of the pet, which the belt is brought into contact with.

Korean Patent No. 1908890 (Dec. 10, 2018) describes a collar for a pet, wherein the collar is configured to enhance identification to others when an owner and a pet walk at night to prevent safety accidents in advance, and to easily find a lost pet through the recognition unit that displays contact information, etc. to prevent disappearance of a pet.

Korean Patent No. 1811011 (Dec. 21, 2017) describes a method for manufacturing a band for a pet. There is a need and a desire for improved pet necklaces.

### BRIEF SUMMARY OF THE EMBODIMENTS

According to a embodiments of the present disclosure, a necklace belt for a pet is provided to include a first belt; and a second belt, wherein the first belt and the second belt are configured to be connected to opposite side surfaces of a wearable sensor device for monitoring a bio-signal of a pet and to be worn around a body part of the pet, and wherein the first belt includes a plurality of fastening holes, and wherein the second belt includes a locking pin configured to be inserted into any one of the plurality of fastening holes and fix the second belt; a through hole into which the first belt is inserted; and a band configured to allow the first belt that has passed through the through hole to be fixed thereby.

Furthermore, each of the first belt and the second belt includes a crease configured to form a groove-shaped crease at an inner portion of a neck of an end thereof connected to the wearable sensor device such that the groove-shaped crease is formed to allow the first and second belt to be bent according to a body shape of the pet.

Furthermore, each of the first belt and the second belt is configured to be fitted to and coupled to a base of the wearable sensor device, and the base and a cover are configured to be fixed by being fastened by a bolt, so that without a tool, separation between the first and second belts and the wearable sensor device is impossible only with repetitive movement of the pet.

Furthermore, each of the first belt and the second belt includes a plurality of holes formed on an end thereof connected to the wearable sensor device, wherein the plurality of holes are configured to be respectively fitted into and fastened to a plurality of pins formed on the base of the wearable sensor device, so that after the base and the cover of the wearable sensor device are fastened to each other, separation there between is prevented.

Furthermore, the base includes a plurality of pins protruding on positions corresponding to the plurality of holes, wherein a protruding length of the plurality of pins is formed longer than a thickness of the first belt and a thickness of the second belt, so that the first and second belts are prevented from being separated from the plurality of pins without vertically raising and removing the first belt and the second belt.

Furthermore, when the base and the cover are fastened to each other, any one of the plurality of pins to which the first belt and the second belt are configured to be fitted and fastened may be inserted into a coupling groove provided at a position corresponding to the cover, so that even when the first belt and the second belt move, the first belt and the second belt are prevented from being separated from the wearable sensor device.

Furthermore, when the base and the cover of the wearable sensor device are securely fastened to each other by a bolt, each of the first belt and the second belt includes an open ring surrounding a circular circumference of the coupling hole formed in the base into which the bolt is coupled, and wherein the open ring is configured to allow the first belt and the second belt to be maintained in a firmly horizontal-fastened state with respect to the wearable sensor device, so that the first belt and the second belt are fixed not to be inserted into and separated from the base.

Furthermore, each of the first belt and the second belt includes a first hole, a second hole, and a third hole configured to be formed on an end thereof connected to the wearable sensor device; and an open ring, wherein when the base and the cover of the wearable sensor device are securely fastened by a bolt, the open ring surround a circular circumference of the coupling hole formed in the base, into which the bolt is coupled, to maintain a firmly fastening state with respect to the wearable sensor device.

Furthermore, each of the first hole and the second hole is configured to be respectively coupled to a first pin and a second pin of the base of the wearable sensor device, the pins being formed by protruding longer than the thickness of each of the first belt and the second belt, and to be formed thin so that when the first belt and the second belt are easily separated from the wearable sensor device when the first belt and the second belt are vertically raised from the wearable sensor device.

Furthermore, the third hole is configured to be coupled to a third pin formed on the base, and to be formed in a long shape so as to stably hold the first and second belts even when the first belt and the second belt are twisted up, down, left and right, and the third hole is configured to be arranged at each vertex of a triangle together with the first hole and the second hole, and the third pin is configured to be formed by protruding from bottom of the base to be longer than the thickness of each of the first belt and the second belt, so that when the base and the cover of the wearable sensor device are fastened to each other, the third pin is configured to be inserted into the coupling groove of the cover.

Furthermore, the open ring is configured to be formed at each of opposite portions of the end of each of the first belt and the second belt, and to prevent the first belt and the second belt from moving transversally, and to prevent the first belt and the second belt from being separated from or inserted into the wearable sensor device.

Furthermore, an end of each of the first belt and the second belt, the end being coupled to the wearable sensor device, is configured to be formed wider than a width of a body of each of the first belt and the second belt, and the open ring is configured to be formed between the body and the end.

In addition, according to an embodiment of present disclosure, a method of configuring a necklace belt for a pet, includes in a system for manufacturing the necklace belt for the pet, configuring a first belt having a plurality of fastening holes; configuring a second belt including a locking pin inserted into any one of the plurality of fastening holes for fixation, a through hole into which the first belt is inserted, and a band through which the first belt that has passes through the through hole is held; and completing the necklace belt for a pet by respectively connecting the provided first and second belts to opposite side surfaces of a wearable sensor device for monitoring a bio-signal of the pet.

Furthermore, each of the configuring of the first belt and the configuring of the second belt includes forming a first hole and a second hole in an end of each of the first belt and the second belt, the end being connected to the wearable sensor device; forming a third hole in the end; and forming an open ring, when a base and a cover of the wearable sensor device are securely fastened to each other by a bolt, the open ring surrounding a circular circumference of a coupling hole of the base, into which the bolt is coupled, and maintaining a firmly fastened state with respect to the wearable sensor device, wherein the first hole and the second hole are configured to be respectively coupled to a first pin and a second pin of the base of the wearable sensor device, the first and second pins being formed by protruding longer than the thickness of each of the first belt and the second belt, and the first hole and the second hole are configured to be formed thin so that the first belt and the second belt are configured to be easily separated from the wearable sensor device when the first belt and the second belt may be vertically raised from the wearable sensor device, and wherein the third hole is configured to be coupled to a third pin formed on the base, and to be formed in a long shape so as to stably hold the first belt and the second belt even when the first belt and the second belt are twisted up, down, left, and right, and the third hole is configured to be arranged at each vertex of a triangle together with the first hole and the second hole, and wherein the open ring is configured to be formed at each of opposite portions of an end of each of the first belt and the second belt so as to prevent the first belt and the second belt from moving transversally, and to prevent the first belt and the second belt from being inserted into and separated from the wearable sensor device.

As described above, according to the necklace belt for a pet of the present disclosure, the necklace belt is connected to each of opposite portions of the wearable sensor device for monitoring a bio-signal of the pet, and when the necklace belt is worn around a body part of the pet by adjusting the length of the necklace belt to fit the body part of the pet, the necklace belt can be easily and firmly bent and locked according to a shape of the body part of the pet, and the necklace belt cannot be overturned on the body part of the pet and there is no risk of separating the necklace belt even with repeated movements.

Other aspects, features, and techniques will be apparent to one skilled in the relevant art in view of the following detailed description of the embodiments.

### BRIEF DESCRIPTION OF THE DRAWINGS

The features, objects, and advantages of the present disclosure will become more apparent from the detailed description set forth below when taken in conjunction with the drawings in which like reference characters identify correspondingly throughout and wherein:

FIG. 1 is a general perspective view including a necklace belt for a pet and the wearable sensor device for monitoring a bio-signal of the pet according to an embodiment of the present disclosure.

FIG. 2 is a perspective view showing a structure of the necklace belt for a pet according to the embodiment of the present disclosure.

FIG. 3 is a detailed view showing a structure of a locking pin according to the embodiment of the present disclosure.

FIG. 4 illustrates a plane view and a side view showing the necklace belt for a pet according to the embodiment of the present disclosure.

FIG. 5 is a view showing, in detail, coupling relationship between a hole provided in the necklace belt for a pet and a pin provided in the wearable sensor device according to the embodiment of the present disclosure.

FIG. 6 is a side sectional view showing, in detail, a structure in a coupled state in which the necklace belt for a pet is coupled to the wearable sensor device according to the embodiment of the present disclosure.

FIG. 7 is a flowchart showing an operation process of a method for configuring the necklace belt for a pet according to the embodiment of the present disclosure.

FIG. 8 is a flowchart showing, in detail, an operation process of the configuration of a first belt of the necklace belt for a pet according to the embodiment of the present disclosure.

### DETAILED DESCRIPTION OF THE EXEMPLARY EMBODIMENTS

### Overview and Terminology

Herein below, an exemplary embodiment of a necklace belt for a pet of the present disclosure will be described with reference to accompanying drawings. Throughout the drawings, the same reference numerals will refer to the same or like elements or parts. Furthermore, the structural or functional description specified to exemplary embodiments of the present disclosure is intended to describe the exemplary embodiment according to the present disclosure. In the flowing description, unless otherwise defined, all terms including technical and scientific terms used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Embodiments are directed to a necklace belt for a pet, and the necklace belt to connect a wearable sensor device for monitoring a bio-signal of a pet to the necklace belt and to allow the necklace belt to be worn around a body part of the pet.

Another objective of a necklace belt for a pet according to the present disclosure is to prevent the necklace belt from being separated from a pet even with repeated movements by coupling a wearable sensor device to the necklace belt such that the necklace belt cannot be separated without a use of a tool by a person.

A further objective of a necklace belt for a pet according to the present disclosure is to prevent the necklace belt from being separated after a base and a cover of a wearable sensor device are fastened to each other by fitting and fastening a pin formed in the base of the wearable sensor device into a hole formed in the necklace belt when the wearable sensor device is coupled to the necklace belt.

A further objective of a necklace belt for a pet according to the present disclosure is to prevent the necklace belt from being separated from a plurality of pins formed in a base of a wearable sensor device, without vertically raising the necklace belt, by forming a protruding length (height) of the plurality of pins longer than a thickness of the necklace belt.

A further objective of a necklace belt for a pet according to the present disclosure is to, when a cover of a wearable sensor device is coupled to a base thereof with the necklace belt coupled to a plurality of pins formed in the base of the wearable sensor device, insert and couple at least one of the plurality of pins into a coupling groove provided in a position corresponding to the cover so as to prevent the necklace belt from being separated from the wearable sensor device, even when the necklace belt moves.

A further objective of a necklace belt for a pet according to the present disclosure is to, when the necklace belt is coupled to a wearable sensor device, form an open ring (fishing hook), so that opposite portions of the necklace belt surround a circular circumference of a coupling hole fastening a base and a cover of the wearable sensor device to each other so as to firmly maintain horizontal-fastened state between the necklace belt and the wearable sensor device, and to lock the necklace belt to prevent the necklace belt from being inserted into and separated from the base of the wearable sensor device.

According to embodiments a necklace belt is provided, and when a wearable sensor device for monitoring a bio-signal of the pet is connected to the necklace belt and is worn to the pet, the necklace belt is capable of being easily and firmly bent according to the body part of the pet and locked, and of being prevented from being overturned on the body part of the pet and from being separated even with repeated movements.

The present disclosure is configured such that a protruding length of at least one pin formed on a base of the wearable sensor device is formed longer than a thickness (height) of the necklace belt to prevent separation between the pin and necklace belt without vertically raising the necklace belt, and when the pin that has been coupled to the necklace belt is coupled to a cover of the wearable sensor device, some pins among the at least one pin enters the inside space of a coupling groove provided at a position corresponding to the cover so that the necklace belt is prevented from being separated from the wearable sensor device even when the necklace belt moves.

On the other hands, since the present disclosure relates to a necklace belt for a pet, wherein a protruding length of at least one pin provided on a base of a wearable sensor device is formed longer than a thickness of the necklace belt so that the necklace is not separated from the pin without being vertically raised and removed, and some pins among the at least one pin is configured to be inserted into a coupling groove provided at a position corresponding to a cover so that the necklace belt is not separated from the wearable sensor device even when the necklace belt moves, the present disclosure has a significant difference in technical configuration over conventional designs.

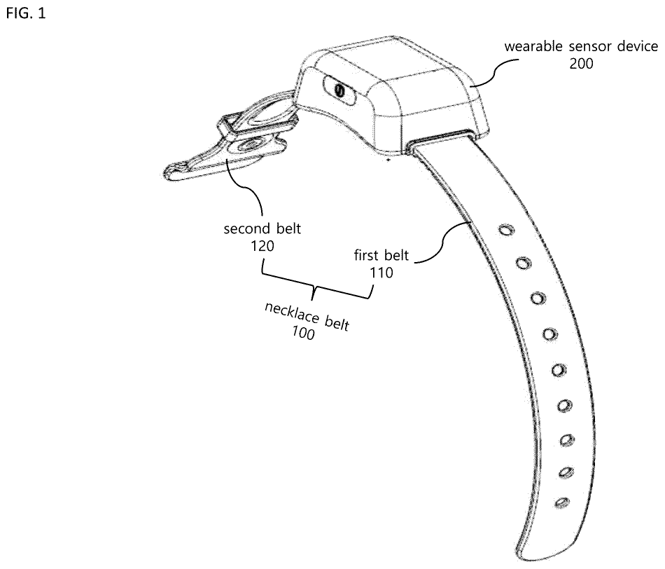

FIG. 1 is a general perspective view including a necklace belt for a pet and the wearable sensor device for monitoring a bio-signal of the pet according to an embodiment of the present disclosure.

As shown in FIG. 1, the present disclosure is a necklace belt 100 for a pet, the necklace belt 100 including a first belt 110 and a second belt 120, and the first and second belt are coupled to opposite side surfaces of a wearable sensor device 200.

When the necklace belt 100 is attached to a pet, the necklace belt 100 coupled to the wearable sensor device 200 is worn around a body part of the pet, and the length of the necklace belt 100 is adjusted to fit to the size of the body part of the pet. Next, the necklace belt 100 is used by fastening the first belt 110 and the second belt 120 to each other. At this time, the necklace belt 100 and the wearable sensor device 200 may be used by being worn around various body parts such as a leg, abdomen, or the like in addition to being worn around the neck of the pet.

Furthermore, when the necklace belt 100 is coupled to the opposite portions of the wearable sensor device 200, the necklace belt is not simply separated due to not only human pulling force, but also repetitive movement of the pet.

The wearable sensor device 200 measures the temperature of the pet, and provides the measured temperature information to a remote server or a mobile terminal of a user who is an owner of the pet via wireless communication such as Bluetooth, Wi-Fi, etc., so that the physical condition of the pet can be monitored in real time. Accordingly, the physical condition of the pet cared by the user can be easily checked, and when an abnormality occurs, the user can take immediate countermeasures, such as taking the pet to a veterinary hospital.

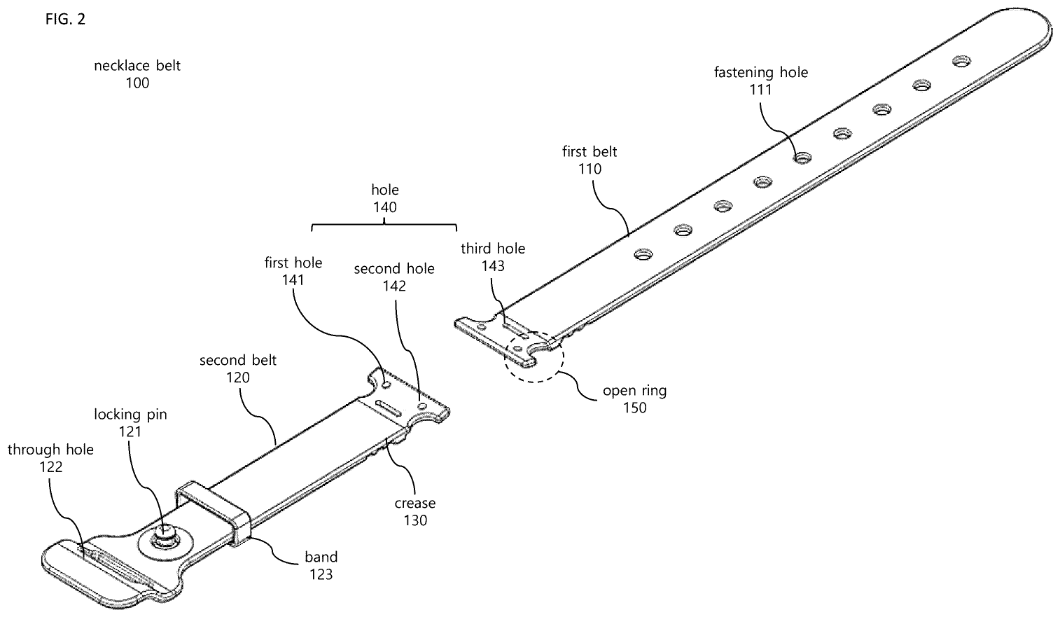

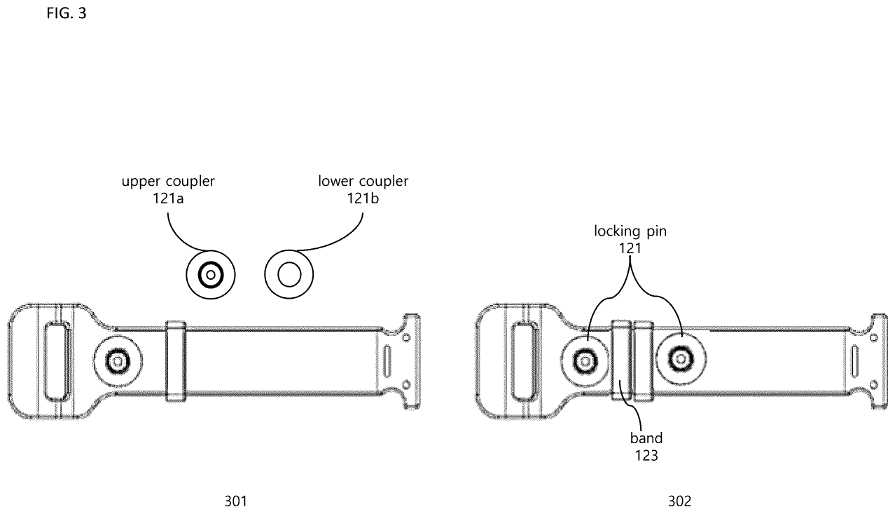

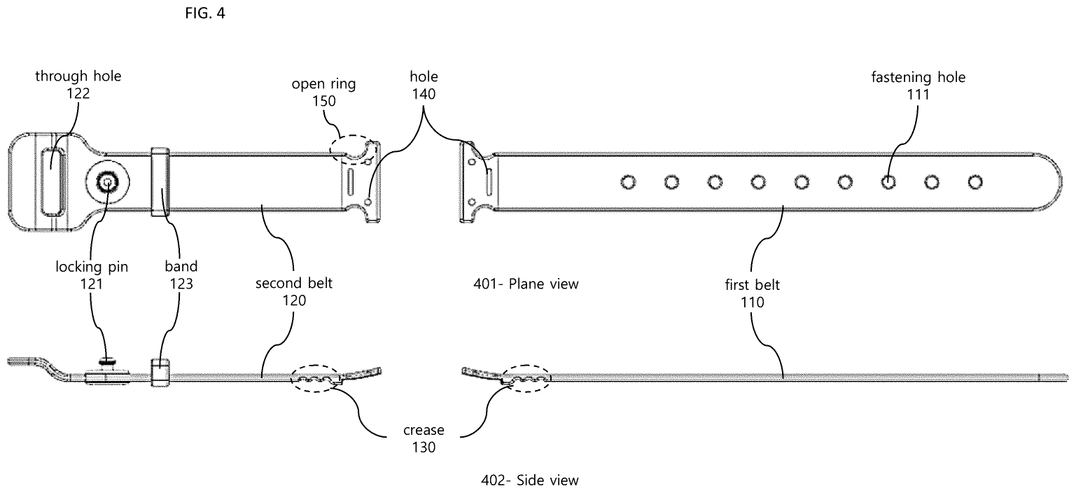

FIG. 2 is a perspective view showing a structure of the necklace belt for a pet according to the embodiment of the present disclosure. FIG. 3 is a detailed view showing a structure of a locking pin according to the embodiment of the present disclosure. FIG. 4 illustrates a plane view 401 and a side view 402 showing the necklace belt for a pet according to the embodiment of the present disclosure.

According to the embodiment of the present disclosure, as shown in FIG. 2, the necklace belt 100 includes the first belt 110, the second belt 120, a crease 130, a plurality of holes 140, an open ring 150, etc. In other words, the necklace belt 100 is configured to be connected to the opposite side surfaces of the wearable sensor device 200 through the first belt 110 and the second belt 120.

The first belt 110 includes a plurality of fastening holes 111, the fastening holes 111 being formed at predetermined intervals as shown in FIGS. 2 and 4.

As shown in FIGS. 2 and 4, the second belt 120 includes a locking pin 121, which is configured such that when the necklace belt 100 is fastened around the a body part of the pet, the first belt 110 is fitted according to the size of the body part of the pet, and then the locking pin 121 is inserted into any one of the plurality of fastening holes 111 and locks the necklace belt 100 in order to maintain the fitted state, a through hole 122 into which the first belt 110 is inserted in a process in which the necklace belt 100 is fastened around the body part of the pet, and a band 123 holding the first belt 110 passing through the through hole 122.

At this point, the button-shaped locking pin 121 is coupled to the second belt 120 made of a material such as silicone, etc. in a fitted manner, so that the more usage the necklace belt is used, the locking pin 121 may be separated from the second belt 120.

Accordingly, in the present disclosure, as shown in 301 of FIG. 3, in order to enhance the coupling force, the locking pin 121 is not formed into an integrated shape, and consists of an upper coupler 121a and a lower coupler 121b. While the upper coupler 121a and the lower coupler 121b are respectively located at an upper surface and a lower surface with the second belt 120 located between the upper coupler 121a and the lower coupler 121b, a protrusion of the upper coupler 121a (i.e., protrusion coupled to the fastening holes 111) is pressed in a downward direction, so that a sharp portion of a lower side of the protrusion may be coupled to the lower coupler 121b in a picked manner.

Furthermore, as shown in 302 of FIG. 3, the locking pin 121 may be provided at each of opposite sides of a double-formed band 123 to change a locking structure into a double locking structure, and in addition to hard plastic material such as silicone, the locking pin 121 may be formed in the form of iron rivets.

Furthermore, at inner portions (lower surfaces in 402 of FIG. 4) of necks of ends of the first belt 110 and the second belt 120, the portions being connected to the wearable sensor device 200, creases 130 having grooves are formed. By creases formed in the crease 130, the ends of the first belt 110 and the second belt 120 are naturally bent according to the shape of the body part of the pet.

Furthermore, as shown in FIGS. 2 and 4, the plurality of holes 140 including a first hole 141, a second hole 142, and a third hole 143 is formed in each of the ends of the first belt 110 and the second belt 120 in common.

The plurality of holes 140 is fitted around pins formed in a base 210 of the wearable sensor device 200. In this state, when the base 210 and a cover 220 are fastened to be fixed by the bolting, without a separate tool, separation by the first belt 110 and the second belt 120 is impossible only with repetitive movement of the pet.

In other words, the first belt 110 and the second belt 120 are coupled to the wearable sensor device 200 such that the holes 140 are respectively fitted around the plurality of pins formed in the base 210 of the wearable sensor device 200. As described above, the first belt 110 and the second belt 120 are fitted and coupled to the base 210 in the pin and hole structure, and then the base 210 and the cover 220 are fixed by being fastened by a bolt. Then, with just simple movement of the pet, the first belt 110 and the second belt 120 are prevented from being separated from the wearable sensor device 200.

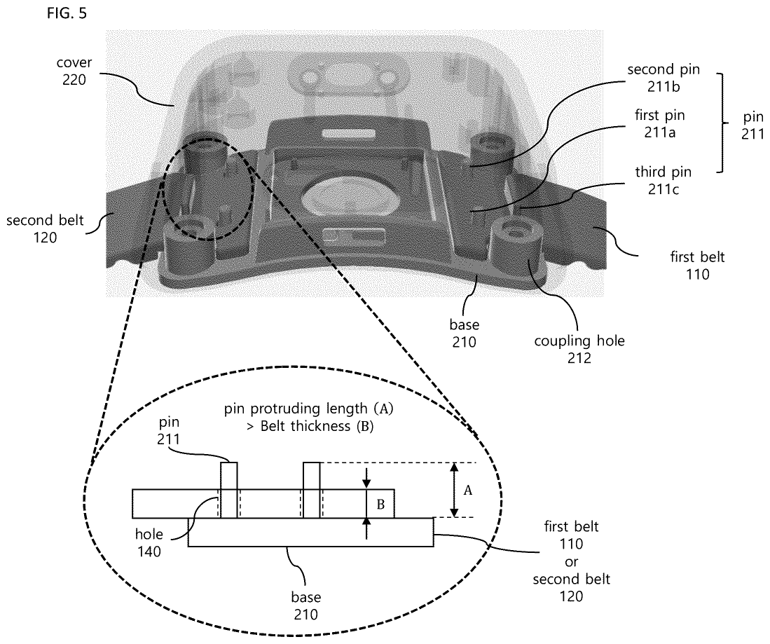

Specifically, the first hole 141 and the second hole 142 are respectively coupled to a first pin 211a and a second pin 211b of the base 210 of the wearable sensor device 200, which are formed by protruding longer than the thickness of the first belt 110 and the thickness of the second belt 120 (referring to FIG. 5).

Furthermore, it is preferable that the first hole 141 and the second hole 142 are formed thin so that when the first belt 110 and the second belt 120 are vertically raised from the wearable sensor device 200, separation thereof is easily performed. In FIGS. 2 and 4, the first hole 141 and the second hole 142 are shown as circular holes.

Furthermore, the third hole 143 is coupled to a third pin 211c formed in the base 210 of the wearable sensor device 200.

At this point, the third hole 143 is formed in a long shape so as to stably lock the first belt 110 and the second belt 120 even when the first belt 110 and the second belt 120 connected to the wearable sensor device 200 are twisted up, down, left, and right, and the third hole 143 is formed to be arranged on each vertex with the first hole 141 and the second hole 142.

Furthermore, as shown in view 401 of FIG. 4, each of the end of the first belt 110 and the end of the second belt 120, the ends being coupled to the wearable sensor device 200, is formed wider than the width of the body of the first belt 110 and the width of the body of the second belt 120, and an open ring 150 is formed on each of opposite sides of a portion between on each end and each body.

As shown in FIGS. 2 and 4, the open ring 150 is formed on each opposite sides of the end of each of the first belt 110 and the second belt 120.

At this point, the open ring 150 is formed to surround a circular circumference of a coupling hole 212 formed on the base 210 to which the bolt is coupled when the base 210 and the cover 220 of the wearable sensor device 200 are fixed by being fastened by a bolt (referring to FIG. 5).

As described above, when the open ring 150 is formed in a shape surrounding the circular circumference of the coupling hole 212 formed in the base 210, transverse movement of the first belt 110 and the second belt 120 can be prevented, and after the first belt 110 and the second belt 120 are coupled to the wearable sensor device 200, separation or entering of the first belt 110 and the second belt 120 can be prevented.

Meanwhile, as shown in FIGS. 2 and 4, one end (i.e., coupled to the base 210 of the wearable sensor device 200) of each of the first belt 110 and the second belt 120, the end having the first hole 141, the second hole 142, the third hole 143, and the open ring 150 may be integrally formed to each of the first belt 110 and the second belt 120 in the same material (e.g., leather, silicone, etc.). Moreover, the end may be made of a material such as stainless, iron, or the like to enhance the strength. Of course, even when the portion with the plurality of holes and the open ring is formed in a different material from a material of the first belt and a material of the second belt, the coupling or inclination between the belts and the wearable sensor device 200 cannot affected therefrom.

FIG. 5 is a view showing, in detail, coupling relationship between a hole provided in the necklace belt for a pet and a pin provided in the wearable sensor device according to the embodiment of the present disclosure.

As shown in FIG. 5, the base 210 of the wearable sensor device 200, of which the opposite sides of the base 210 are coupled to the necklace belt 100, has the plurality of pins 211 and a plurality of coupling holes 212.

The plurality of pins 211 includes the first pin 211a, the second pin 211b, and the third pin 211c, the pins being formed by protruding from positions corresponding to the first hole 141, the second hole 142, and the third hole 143 as shown in FIG. 2.

At this point, the first pin 211a and the second pin 211b are formed into cylindrical shapes so as to be inserted into the first hole 141 and the second hole 142 that are formed into circular shapes as shown in FIG. 2, and the third pin 211c is formed into a vertically standing plate such that the long form of the third hole 143 is fitted around. Accordingly, even when the first belt 110 and the second belt 120 coupled to the opposite sides of the wearable sensor device 200 are twisted in various directions, it is possible to stably hold the first belt and the second belt.

Specifically, in the present disclosure, the protruding length (A) of the plurality of pins 211 is formed longer than the thickness (B) of the first belt 11 and the second belt 120, whereby the first belt 11 and the second belt 120 cannot be separated from the plurality of pins 211 without vertically raising the first belt 110 and the second belt 120.

The coupling hole 212 is formed in each corner of the base 210, and is a portion where the base 210 and the cover 220 are fastened to each other by a bolt.

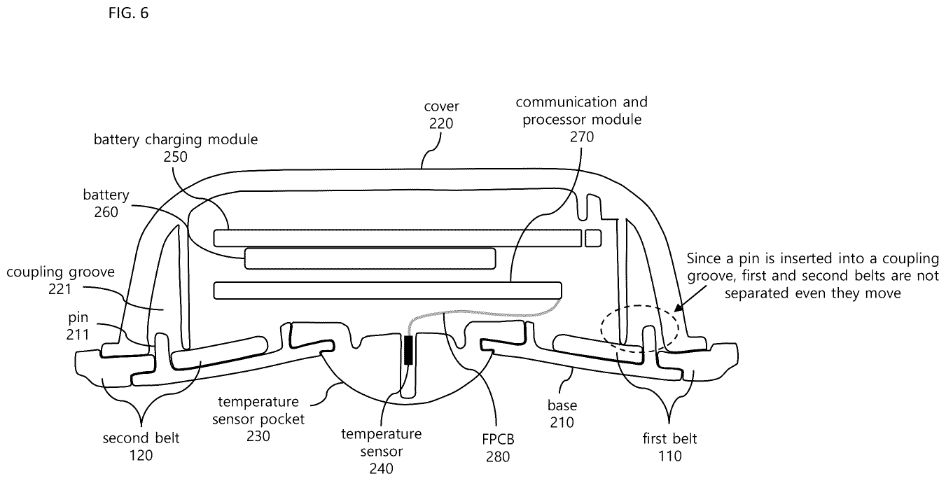

FIG. 6 is a side sectional view showing, in detail, a structure in a coupled state in which the necklace belt for a pet is coupled to the wearable sensor device according to the embodiment of the present disclosure.

As shown in FIG. 5, the third pin 211c provided on the base 210 of the wearable sensor device 200 is formed by protruding from the bottom of the base 210 to be longer than the thickness of the first belt 110 and the thickness of the second belt 120.

Therefore, as shown in FIG. 6, after the first belt 110 and the second belt 120 are respectively fitted and coupled to the base 210 of the wearable sensor device 200, in a process in which the base 210 and the cover 220 are fastened to each other, an upper portion of the third pin 211c is inserted into a coupling groove 221 formed in the cover 220.

As described above, when the upper portion of the third pin 211c is inserted into the coupling groove 221 formed in the cover 220, even when the first belt 110 and the second belt 120 move, the first belt 110 and the second belt 120 is never separated from the wearable sensor device 200.

Meanwhile, as shown in FIG. 6, the wearable sensor device 200 includes the base 210, the cover 220, a temperature sensor pocket 230, a temperature sensor 240, a battery charging module 250, a battery 260, a communication and processor module 270, a flexible print circuit board (FPCB) 280, and the like.

The base 210 constitutes a lower portion of the wearable sensor device 200 to monitor a bio-signal (e.g., body temperature) of the pet.

At this point, a portion of the base 210 in contact with the body part of the pet is preferably formed in a streamlined-curved shape. The shape is intended to naturally fasten the wearable sensor device 200 in response to the body flexion of the pet.

The cover 220 is formed in a cap shape in which a space is provided and is coupled to an upper portion of the base 210, and serves to protect the wearable sensor device 200 to monitor the bio-signal of the pet.

In other words, the printed circuit board including the battery charging module 250, the battery 260, and the communication and processor module 270 is securely installed in the inner space, and the temperature sensor 240 is inserted into and coupled to the temperature sensor pocket 230 via the FPCB 280 connected to one portion of the communication and processor module 270.

The temperature sensor pocket 230 is a portion where the temperature sensor 240 for measuring the temperature of the pet is mounted, and is coupled to the base 210 in a top-to-bottom direction.

Specifically, the temperature sensor pocket 230 prevents the temperature sensor 240 from being brought directly into contact with the skin of the pet, and in order to transmit the body temperature of the pet to the temperature sensor 240, the temperature sensor pocket 230 is coupled to the base 210 in the shape protruding downward of the base 210.

Furthermore, the temperature sensor pocket 230 is made of eco-friendly silicone material to prevent trouble even when the wearable sensor device 200 is brought into contact with the skin of the pet, and to improve thermal conductivity so that the temperature sensor 240 can precisely measure the body temperature of the pet.

The temperature sensor 240 senses the body temperature of the pet, and is inserted into and installed at a temperature sensor insertion port provided in the temperature sensor pocket. The temperature sensor 240 measures the body temperature of the pet by thermal transfer via the temperature sensor pocket without contacting the skin of the pet, and provides the measured data for the measured body temperature of the pet to the communication and processor module 270.

The battery charging module 250 controls power, which is provided from the outside space via a power cable connected to a charging terminal provided at one portion of the cover 220, so that the battery 260 is charged with the power.

The battery 260 is charged with power supplied from the outside space according to the control of the battery charging module 250, and according to a control of the communication and processor module 270, the battery 260 supplies the charged power for each configuration of the operation of the wearable sensor device 200.

The communication and processor module 270 includes an antenna and a wireless communication transceiver for the near-field communication. The bio-signal (i.e., body temperature) of the pet measured by the temperature sensor 240 is transmitted to the mobile terminal of the user via the antenna, or is transmitted to a server of a cloud via wireless communication transceiver, and the server of the cloud may transmit the bio-signal of the pet to the mobile terminal of the user who is the owner of the pet.

The FPCB 280 includes a power line and a signal line, and connected to the temperature sensor 240 and the communication and processor module 270 while being located therebetween.

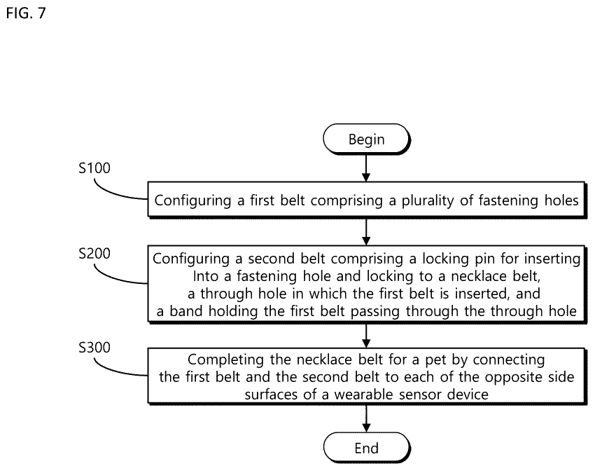

Next, as described above, a method of configuring the necklace belt for a pet will be described in detail according to FIGS. 7 and 9. Here, the order of each stage according to the method of the present disclosure may be changed depending on the use environment or a person skilled in the art.

FIG. 7 is a flowchart showing an operation process of a method for configuring the necklace belt for a pet according to the embodiment of the present disclosure.

As shown in FIG. 7, in a system (not shown) for manufacturing the necklace belt for a pet, the method includes configuring, at S100, the first belt 110 including the plurality of fastening holes 111 as shown in FIG. 2.

Furthermore, the method includes configuring, at S200, the second belt 120 including the locking pin 121 inserted into any one of the plurality of fastening holes 111 to lock the necklace belt 100, the through hole 122 into which the first belt 110 is inserted, and the band 123 holding the first belt 110 passing through the through hole 122.

Next, the method includes completing, at S300, the necklace belt for a pet by connecting the first belt 110 provided through the stage S100 and the second belt 120 provided through the stage S200 to the opposite side surfaces of the wearable sensor device 200 to monitor the bio-signal of the pet.

The necklace belt 100 for a pet and the wearable sensor device 200, which are completed as described above, are mounted to the body part of the pet to be monitored, and the wearable sensor device 200 measures the body temperature of the pet according to a preset period. Next, the information about the measured body temperature is provided to a remote server or a mobile terminal of a user, who is an owner of the pet via wireless communication such as Bluetooth, Wi-Fi, etc. Accordingly, the user, who is the owner of the pet, monitors the physical condition of the pet transmitted from the wearable sensor device 200, and when an abnormality occurs, the user can take immediate countermeasures.

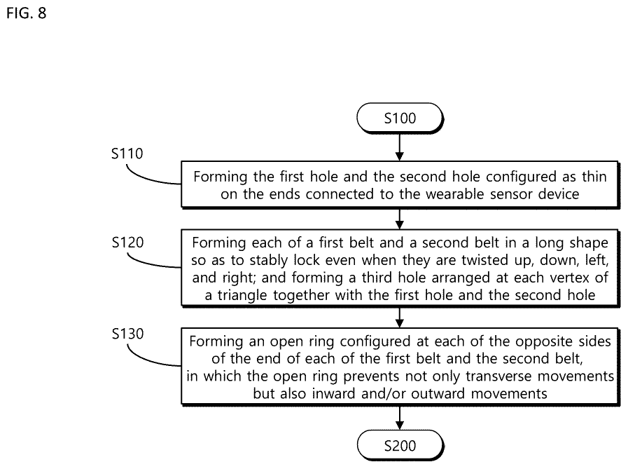

FIG. 8 is a flowchart showing, in detail, an operation process of the configuration of a first belt of the necklace belt for a pet according to the embodiment of the present disclosure.

As shown in FIG. 8, in the configuring at S200 of the first belt 110 is performed by forming, at S110, the first hole 141 and the second hole 142 on the end of the first belt 110 connected to the wearable sensor device 200, and then is performed by forming, S120, the third hole 143 on the end.

In forming S110 of the first hole 141 and the second hole 142, the first hole 141 and the second hole 142 are formed thin so as to be respectively coupled to the first pin 211a and the second pin 211b of the base 210 of the wearable sensor device 200, the first pin 211a and the second pin 211b being formed by protruding longer than the thickness (height) of the first belt 110 and the thickness (height) of the second belt 120.

As described above, by forming the first hole 141 and the second hole 142 thin, separation of the first belt 110 and the second belt 120 is easily performed when the first belt 110 and the second belt 120 are vertically raised from the wearable sensor device 200.

Furthermore, in the forming S120 of the third hole 143, the third hole 143 is formed in a long shape so as to stably lock each of the first belt 110 and the second belt 120 even when the first belt 110 and the second belt 120 are twisted up, down, left, and right. Simultaneously, the third hole 143 should be arranged at each vertex of a triangle together with the first hole 141 and the second hole 142 formed in the forming at S110.

After the forming S110 and S120 of the plurality of holes, when the base 210 and the cover 220 of the wearable sensor device 200 are fixed to each other by being fastened by a bolt, the method includes forming, at S130, the open ring 150 surrounding the circular circumference of the coupling hole 212, into which the bolt is coupled, formed in the base 210 and maintaining a solid fastening state with respect to the wearable sensor device 200.

At this point, as shown in FIG. 2, the open ring 150 is formed at each of the opposite portions of the end of each of the first belt 110 and the second belt 120. Therefore, not only transverse movements of the first belt 110 and the second belt 120 but also movements of the first belt 110 and the second belt 120 separated from the wearable sensor device 200 are prevented.

Meanwhile, as described in FIG. 8, the method of configuring the holes 140 including the first hole 141, the second hole 142, and the third hole 143, and the open ring 150 is applied equal to the second belt 120, and the detailed description thereof will be omitted since the description is the same as FIG. 8.

As described above, according to the present disclosure, the necklace belt is connected to each of opposite portions of the wearable sensor device for monitoring a bio-signal of the pet, and when the necklace belt is worn around the body part of the pet by adjusting the length of the necklace belt to fit the body part of the pet, the necklace belt can be easily and firmly bent and locked according to a shape of the body part of the pet, and the necklace belt cannot be overturned on the body part of the pet and there is no risk of separating the necklace belt even with repeated movements.

As described above, the present disclosure have been described with reference to the embodiment shown in the accompanying drawings, but described for illustrative purposes, and those skilled in the art will understand that various modifications, additions and substitutions are possible, without departing from the scope and spirit of the present disclosure as disclosed in the accompanying claims. Therefore, the patent right of the present disclosure should be defined by the scope and spirit of the invention as disclosed in the accompanying claims.

What is claimed is:

1. A necklace belt for a pet, the necklace belt comprising:
a first belt; and
a second belt,
wherein the first belt and the second belt are configured to be connected to opposite side surfaces of a wearable sensor device for monitoring a bio-signal of a pet and to be worn around a body part of the pet, and
wherein the first belt includes a plurality of fastening holes, and
wherein the second belt includes a locking pin configured to be inserted into any one of the plurality of fastening holes and fix the second belt;
a through hole into which the first belt is inserted; and
a band configured to allow the first belt that has passed through the through hole to be fixed thereby.

2. The necklace belt of claim 1, wherein each of the first belt and the second belt includes
a crease configured to form a groove-shaped crease at an inner portion of a neck of an end thereof connected to the wearable sensor device such that the groove-shaped crease is formed to allow the first and second belt to be bent according to a body shape of the pet.

3. The necklace belt of claim 1, wherein each of the first belt and the second belt is configured to be fitted to and coupled to a base of the wearable sensor device, and the base and a cover are configured to be fixed by being fastened by a bolt, so that without a tool, separation between the first and second belts and the wearable sensor device is impossible only with repetitive movement of the pet.

4. The necklace belt of claim 1, wherein each of the first belt and the second belt includes a plurality of holes formed on an end thereof connected to the wearable sensor device,
wherein the plurality of holes are configured to be respectively fitted into and fastened to a plurality of pins formed on the base of the wearable sensor device, so that after the base and the cover of the wearable sensor device are fastened to each other, separation there between is prevented.

5. The necklace belt of claim 4, wherein the base includes a plurality of pins protruding on positions corresponding to the plurality of holes,
wherein a protruding length of the plurality of pins is formed longer than a thickness of the first belt and a thickness of the second belt, so that the first and second belts are prevented from being separated from the plurality of pins without vertically raising and removing the first belt and the second belt.

6. The necklace belt of claim 5, wherein when the base and the cover are fastened to each other, any one of the plurality of pins to which the first belt and the second belt are configured to be fitted and fastened may be inserted into a coupling groove provided at a position corresponding to the cover, so that even when the first belt and the second belt move, the first belt and the second belt are prevented from being separated from the wearable sensor device.

7. The necklace belt of claim 1, wherein when the base and the cover of the wearable sensor device are securely fastened to each other by a bolt, each of the first belt and the second belt includes an open ring surrounding a circular circumference of the coupling hole formed in the base into which the bolt is coupled, and
wherein the open ring is configured to allow the first belt and the second belt to be maintained in a firmly horizontal-fastened state with respect to the wearable sensor device, so that the first belt and the second belt are fixed not to be inserted into and separated from the base.

8. The necklace belt of claim 1, wherein each of the first belt and the second belt includes a first hole, a second hole, a third hole configured to be formed on an end thereof connected to the wearable sensor device; and an open ring,
wherein when the base and the cover of the wearable sensor device are securely fastened by a bolt, the open ring surround a circular circumference of the coupling hole formed in the base, into which the bolt is coupled, to maintain a firmly fastening state with respect to the wearable sensor device.

9. The necklace belt of claim 8, wherein each of the first hole and the second hole is configured to be respectively coupled to a first pin and a second pin of the base of the wearable sensor device, the pins being formed by protruding longer than the thickness of each of the first belt and the second belt, and to be formed thin so that when the first belt and the second belt are easily separated from the wearable sensor device when the first belt and the second belt are vertically raised from the wearable sensor device.

10. The necklace belt of claim 8, the third hole is configured to be coupled to a third pin formed on the base, and to be formed in a long shape so as to stably hold the first and second belts even when the first belt and the second belt are twisted up, down, left and right, and the third hole is configured to be arranged at each vertex of a triangle together with the first hole and the second hole, and
the third pin is configured to be formed by protruding from bottom of the base to be longer than the thickness of each of the first belt and the second belt, so that when the base and the cover of the wearable sensor device are fastened to each other, the third pin is configured to be inserted into the coupling groove of the cover.

11. The necklace belt of claim 8, wherein the open ring is configured to be formed at each of opposite portions of the end of each of the first belt and the second belt, and
to prevent the first belt and the second belt from moving transversally, and
to prevent the first belt and the second belt from being separated from or inserted into the wearable sensor device.

12. The necklace belt of claim 8, wherein an end of each of the first belt and the second belt, the end being coupled to the wearable sensor device, is configured to be formed wider than a width of a body of each of the first belt and the second belt, and
the open ring is configured to be formed between the body and the end.

13. A method of configuring a necklace belt for a pet, the necklace belt comprising:
in a system for manufacturing the necklace belt for the pet, configuring a first belt having a plurality of fastening holes;
configuring a second belt including a locking pin inserted into any one of the plurality of fastening holes for fixation, a through hole into which the first belt is inserted, and a band through which the first belt that has passes through the through hole is held; and
completing the necklace belt for a pet by respectively connecting the provided first and second belts to opposite side surfaces of a wearable sensor device for monitoring a bio-signal of the pet.

14. The method of claim 13, wherein each of the configuring of the first belt and the configuring of the second belt includes
forming a first hole and a second hole in an end of each of the first belt and the second belt, the end being connected to the wearable sensor device;
forming a third hole in the end; and
forming an open ring, when a base and a cover of the wearable sensor device are securely fastened to each other by a bolt, the open ring surrounding a circular circumference of a coupling hole of the base, into which the bolt is coupled, and maintaining a firmly fastened state with respect to the wearable sensor device,
wherein the first hole and the second hole are configured to be respectively coupled to a first pin and a second pin of the base of the wearable sensor device, the first and second pins being formed by protruding longer than the thickness of each of the first belt and the second belt, and the first hole and the second hole are configured to be formed thin so that the first belt and the second belt are configured to be easily separated from the wearable sensor device when the first belt and the second belt may be vertically raised from the wearable sensor device, and
wherein the third hole is configured to be coupled to a third pin formed on the base, and to be formed in a long shape so as to stably hold the first belt and the second belt even when the first belt and the second belt are twisted up, down, left, and right, and the third hole is configured to be arranged at each vertex of a triangle together with the first hole and the second hole, and
wherein the open ring is configured to be formed at each of opposite portions of an end of each of the first belt and the second belt so as to prevent the first belt and the second belt from moving transversally, and to prevent the first belt and the second belt from being inserted into and separated from the wearable sensor device.
